(19) United States
(12) Patent Application Publication
(10) Pub. No.: US 2008/0058236 A1
(43) Pub. Date: Mar. 6, 2008

(54) Soap Bar Compositions Comprising Alpha Sulfonated Alkyl Ester or Sulfonated Fatty Acid and Synthetic Surfactant and Process for Producing the Same

(76) Inventors: Carlos E. Ospinal, Streamwood, IL (US); Branko Sajic, Lincolnwood, IL (US); Xue Min Dong, Lincolnshire, IL (US)

Correspondence Address: MCANDREWS HELD & MALLOY, LTD, 500 WEST MADISON STREET, SUITE 3400, CHICAGO, IL, 60661, US

(21) Appl. No.: 11/929,062

(22) Filed: Oct. 30, 2007

Related U.S. Application Data
(63) Continuation of application No. 11/436,280, filed on May 18, 2006.
Application No. 11/436,280 is a continuation-in-part of application No. 11/006,968, filed on Dec. 8, 2004, now abandoned.
Application No. 11/006,968 is a continuation-in-part of application No. 10/502,915, filed on Dec. 8, 2004, now abandoned.
(60) Provisional application No. 60/353,693, filed on Jan. 31, 2002.

(51) Int. Cl. A61K 8/02

(52) U.S. Cl. 510/141; 510/152

(57) ABSTRACT

A composition suitable for use in personal cleaning or detergent soap bars, which includes a primary surfactant comprising a sulfonated fatty acid, an alpha sulfonated alkyl ester, or a mixture thereof, and a secondary synthetic surfactant, and methods for producing such a composition. The composition and methods exhibit efficient processing and allow for formation of cleansing or detergent bars with improved hardness, improved resistance to marring, improved processability, lower wear-rate and decreased mush formation during consumer use.

This application is a continuation-in-part of pending U.S. applicaiton Ser. No. 11/006,968, filed Dec. 8, 2004, which is a continuation-in-part of pending U.S. application Ser. No. 10/502,915, filed Dec. 8, 2004, which is a national phase application of PCT/US03/02861, filed Jan. 31, 2003, which claims priority to U.S. Provisional App. Ser. No. 60/353693, filed Jan. 31, 2002 (now abandoned), each of which are incorporated by reference in their entirety.

### FIELD OF THE INVENTION

This presently described technology relates to compositions comprising a soap, a fatty acid, a primary surfactant comprising sulfonated fatty acid, alpha sulfonated alkyl ester, or a mixture thereof, a secondary synthetic surfactant, an electrolyte and a polyhydric alcohol, wherein said compositions are suitable for formation into precursor cleansing/laundry bar pre-blends (i.e., “soap noodles”), finished personal cleansing bars, or finished laundry detergent bars. Specifically, the invention relates to compositions suitable for processing into solid or semi-solid personal cleansing and/or laundry detergent bars that contain α-sulfonated fatty acid alkyl ester and/or sulfonated fatty acid in combination with at least one synthetic anionic, amphoteric, zwitterionic, nonionic, or semi-polar surfactant. The presently described technology additionally relates to an improved process for producing such precursor cleansing/laundry bar surfactant pre-blends or personal cleansing/laundry detergent bars. Embodiments of the present compositions and processes exhibit improved processing characteristics and allow for formation of cleaning or detergent bars with improved hardness, improved resistance to marring, lowered wear-rate and decreased mush formation during consumer use.

### DESCRIPTION OF THE RELATED ART

Personal cleansing and laundry cleaning bars, and their precursor formulations, have become a focus of great interest. People generally wash and exfoliate their skin with various surface-active detergent bar formulations several times a day. Ideal skin cleansing bars should cleanse the skin gently, causing little or no irritation, without de-fatting and over-drying the skin or leaving it taut after frequent routine use. Most high lathering soap bars fail in this respect.

The processability, firmness, smearing and marring properties of personal cleansing and laundry cleaning bars as well as the processability of their precursor detergent compositions has become a focus of great interest to the personal care and laundry industries. Precursor cleansing/laundry bar surfactant pre-blends which have lower viscosities and are easily extruded and plodded are highly desirable. Final bars which are easily processed from such precursor compositions which are also very mild, firm but not hard, have low smear and do not readily mar are also highly desirable.

Synthetic detergent bars, frequently called “combo bars” (i.e., a bar having substantial amounts of soap) and/or “syndet bars” (i.e., a bar having very little or no soap) are well known to the art, along with natural “soap” bars for personal care use. Syndet bars often possess poor physical properties, e.g., they exhibit off odors, poor processability, stickiness, brittleness, bar mushiness, poor lather quality, lack of mildness or combinations thereof. Additionally, the problems of formulating synthetic detergent bars are not limited to the performance characteristics of the finished bars. Most synthetic bars which are made with certain mild surfactants are very difficult to fabricate. Processing conditions for such bars present relatively high technical challenges to commercial scale manufacturers, primarily due to the need of expensive special handling equipment.

In contrast, the fabrication of relatively pure “soap” bars is a well defined engineering procedure involving milling, plodding and molding. For example, coco/tallow soap becomes quite plastic when warmed and can be easily plodded and molded under relatively low pressures. However, most synthetic detergents and detergent-filler compositions for use in cleansing or laundry detergent bars become overly plastic and pasty and the machinery for fabrication and processing is often complicated and must be specially designed. See, e.g., U.S. Pat. No. 2,678,921, to Turek et al., issued on May 18, 1954. Ideally, processing of syndet bars or synthetic detergent bars should be fast and problem free in terms of milling, extruding, plodding, molding and stamping of the finished bar. Most mild syndet bar processes fall short in some or all of these respects.

Synthetic detergent bar formulations for personal care use are well known to the art. For example, see U.S. Pat. No. 5,328,632, to Redd et al., issued on Jul. 12, 1994; U.S. Pat. No. 5,510,050, to Dunbar et al., issued on Apr. 23, 1996; U.S. Pat. No. 5,393,449, to Jordan et al., issued on Feb. 28, 1995; WO 95/27036, to Fakoukakis et al., published on Oct. 12, 1995; and WO 95/27038, to Fakoukakis et al., published on Oct. 12, 1995. The major drawbacks of most synthetic surfactant toilet bar formulations include poor lather, poor smear, and poor processability due to stickiness. The use of high lathering anionic surfactants can yield acceptable lather volume, but unfortunately, the use of high lathering anionic surfactants does, in fact, lead to poor processability. While some known mild blends of sodium coconut/tallow alkyl glyceryl ether sulfonate (AGS) are relatively good in lather potential, they are difficult to process because of their stickiness or hygroscopic nature. It will be appreciated that processability, firmness, smear, low marring, mildness, lather, and rinsability make surfactant selection and stoichiometry of ingredients for mild personal cleansing bars a critical and difficult task. Thus, it will also be appreciated that rather stringent requirements for formulating mild personal cleansing bars limit the choice of surfactants, and final formulations represent some degree of compromise. Mildness is often obtained at the expense of processability, effective cleansing, lathering, or rinsing, and vice versa. Processability is often obtained at the expense of smear or marring of the finished bar.

Synthetic detergent bar formulations for laundry cleaning are also well known. Some examples include U.S. Pat. No. 5,965,508, to Ospinal et al., issued on Oct. 12, 1999; WO 95/27036, to Fakoukakis et al., published on Oct. 12, 1995; and WO 95/27038, to Fakoukakis et al., published on Oct. 12, 1995. Such laundry detergent bars have found expanded use in regions of the world where automatic clothes washing machines are not common. The ideal laundry detergent bar is effective in cleaning clothes, has acceptable lathering characteristics, low smear, and pleasing odor and appearance. As these laundry detergent bars are in contact with the skin during clothes washing, mildness is also highly desirable.

Methods for making laundry detergent bars are also known. Some examples include Philippine Pat. No. 23,689, to Kenyon et al., issued on Sep. 27, 1989; and Philippine Pat. No. 24,551, to McGee et al., issued on Aug. 3, 1990. Much like the syndet bars for personal care use, laundry detergent bars often possess many of the same physiochemical problems, e.g., harshness, poor lather, poor smear, poor marring and poor processability due to stickiness.

Conventionally milled toilet soaps are made by a process which generally comprises (1) drying soap having a moisture content of from about 28% to about 30% down to a moisture content of about 7% to about 14%, (2) forming the dried soap into precursor “soap noodles,” by passing it through a plodder, (3) mixing the various desired additives such as colorants, perfume, etc., into the soap noodles, (4) passing the mixture formed in (3) through a mill or series of mills (“milling” the soap) thereby forming ribbons of soap, (5) passing the milled soap mixture from (5) through another plodder to form a log of soap (i.e., “plodding” the soap to form a “billet”), and (6) cutting the log into segments (i.e., billets) and stamping the segments or “billets” into the desired bar shape.

The soap which is dried in step (1) can generally be made from saponification of fats or neutralization of free fatty acids. Because the drying is never completely uniform, the dried soap inevitably contains some particles which are over-dried and are harder than the remaining bulk of the dried soap. If the soap also contains free fatty acid, non-homogeneity of the free acid in the soap can also contribute to the presence of soap particles which are harder than the remaining bulk of the dried soap. The hard particles are generally from about 0.5 to about 10 mm in diameter. These particles remain in the soap through the first plodding step (2) and the mixing step (3). In the milling step (4), the soap is “worked” and the over-dried particles are broken down into much smaller particles (generally less than about 0.25 mm in diameter) and are homogeneously distributed throughout the soap mass. In the absence of milling, the finished bar may exhibit a rough or sandy feel during use, due to the slower dissolution rate of the relatively large over-dried soap particles, also called “hard specks.” When the soap has been properly milled, the over-dried soap cannot be detected during use, because it has been reduced to a much smaller particle size and is distributed uniformly throughout the soap mass. See British Pat. No. 512,551, to Fairweather, issued on Sep. 19, 1939, incorporated herein by reference; and U.S. Pat. No. 4,405,492 to Nyquist et al., issued on Sep. 20, 1983.

Mild, detergent-soap, and toilet bars containing C₆-C₁₈ acyl isethionate as the principal detergent and minor amounts of fatty acids and soap are disclosed in U.S. Pat. No. 2,894,912 ('912 patent), to Geitz, issued on Jul. 14, 1959; and U.S. Pat. No. 3,376,229 ('229 patent), to Haass et al., issued on Apr. 2, 1968. In the '912 patent, the chips processed into bars are produced from either a 40-50% aqueous slurry of the ingredients mixed at a temperature of from 38° C. to 93° C., or from a mixture of the dry ingredients mixed at 100° C. for a long period of time. In the '229 patent, the bars are prepared from a liquid mixture of acyl isethionate, fatty acids, anionic syndet and soap mixed at a temperature of about 110° C. to 113° C. for about fifteen minutes. The latter bars contain at least about 4% by weight of sodium isethionate as a processing aid.

In U.S. Pat. No. 4,707,288, to Irlam et al., issued on Nov. 17, 1987, mixtures of acyl isethionate, fatty acids, soap and more than 2% by weight of sodium isethionate are mixed in particulate form at temperatures in the range of 60° C. to 86° C. using a special cavity transfer mixer under conditions of high shear to yield toilet bars which exhibit reduced grit.

U.S. Pat. No. 4,696,767, to Novakovic, issued on Sep. 29, 1987, discloses a process for making mild toilet bars wherein a slurry of acyl isethionate, water and a polyol such as sorbitol is formed into a stable solution by heating at a temperature of from 100° C. to 120° C. at 4-10 p.s.i.g. The slurry is then mixed with neat soap and is heated to about 150° C. under a pressure of 4 atmospheres before being spread through a vacuum drying and plodding step to provide flakes which yield a toilet bar without grit. However, the presence of the polyol leads to increased water penetration in the soap dish as well as a bar of increased cost. This patent further provides that use of acyl isethionate in particulate form causes problems, such as lacrimation (i.e., the weeping of material out of the soap bar). Further, larger particles of acyl isethionate yield bars with grit.

In U.S. Pat. No. 4,663,070, to Dobrovolny et al., issued on May 5, 1987, a toilet bar composition in which soap is the principal surfactant is described. Liquid mixtures containing a major proportion of soap plus acyl isethionate, fatty acids, water and sodium isethionate were formed at temperatures of 96° C. to 103° C. In U.S. Pat. No. 5,030,376, to Lee et al., issued on Jul. 9, 1991, a similar mixture containing a major proportion of soap is processed under conditions of high shear in a special cavity transfer mixer at temperatures maintained below 40° C. to form a mixture with some of the soap in the delta phase. U.S. Pat. No. 5,041,233, to Kutny et al., issued on Aug. 20, 1991, also relates to a similar mixture wherein a mixture of acyl isethionate, fatty acids and soap is prepared at a temperature of 82° C. to 94° C., with the soap being formed in situ. This patent indicates that high viscosity mixtures and hydrolysis of acyl isethionate and leads to problems in the final product.

The foregoing description of the relevant art indicates that a variety of processes have been employed to produce personal cleansing and laundry detergent bar pre-bends and the resulting mild, detergent-soap, toilet bars. Further, soap bars are commercially manufactured in a variety of aesthetically pleasing configurations. These products are frequently damaged by marring which is defined as the formation of undesirable, white, chalk-like shatter marks in and around dented areas on conventional soaps. Marring typically occurs during handling, shipping and distribution of finished products to customers.

Approximately one to two weeks after soap bar preparation, ordinary gift and decorative soaps bruise and chip especially on the edges and corners of intricate or unique configurations. When soap products are packed side-by-side, marring often occurs because individual bars bump against each other or against carton partitions and side walls. This marring is readily noticed, especially with colored soap where the chalk-like marks form around the bruises and chips.

Labor intensive packaging processes are currently used to protect conventional soap bases against marring. Novelty products which depend heavily on aesthetically pleasing qualities have previously required expensive cartons and/or protective wrappings to prevent surface defects. Even with these extra precautions, there is no guarantee that conventional formulations will avoid surface defects.

Thus, based on the foregoing, a need exists for superior personal cleansing and/or laundry detergent bar formulations which exhibit enhanced mildness, improved processability, reduced smear, improved lather potential, improved rinsability, and low marring characteristics.

### SUMMARY OF THE INVENTION

Accordingly, the present technology overcomes one or more of the foregoing disadvantages of conventional soap bar compositions and processes by exhibiting surprising performance and processing synergies. Specifically, based on surprising and unique synergism discovered between the component compounds of the present technology, compositions of the present technology are useful in as precursor cleansing/laundry bar surfactant pre-blends or “soap noodles,” finished personal cleansing bars, or finished laundry detergent bars. Soap compositions produced according to embodiments of the present technology generally exhibit improved processability. Bars produced according to embodiments of the present technology generally also exhibit increased foaming properties, decreased smear properties, decreased marring properties, improved color stability, and/or impart superior feel and after-feel properties to skin. Furthermore, the compositions may be translucent and/or can be processed into translucent personal cleansing and/or laundry detergent bars with the appropriate choice of additional components. The compositions are preferably generally suitable for processing using standard extrusion and/or plodder equipment.

Preferably, compositions according to the present technology comprise: a soap, preferably tallow and/or coconut soap; a primary surfactant comprising an alpha sulfonated alkyl ester, sulfonated fatty acid, and/or mixtures thereof; a C₆-C₂₂ fatty acid, an electrolyte (salt), a polyhydric alcohol, and water. Embodiments of the invention may additionally comprise one or more secondary synthetic anionic, amphoteric, zwitterionic, nonionic, or semi-polar surfactants.

It has been surprisingly discovered that the use of a polyhydric alcohol in combination with an electrolyte and an alpha sulfonated alkyl ester, sulfonated fatty acid, and/or a mixture thereof, greatly facilitates and improves the production of precursor cleansing/laundry bar “soap noodles” and personal cleansing/laundry detergent bars prepared from such noodles. The bars generally contain very low moisture levels, thus improving bar hardness properties and lowering wear rates during use. The compositions of the instant invention exhibit lower processing viscosities, improved drying characteristics, and are substantially free of gritty feel caused by the presence of hard particles of soap (“hard specks”), as compared to traditional bar compositions which are substantially free of polyhydric alcohols.

Furthermore, the compositions are useful in preparing stamped, personal cleansing and/or laundry detergent bars which generally have improved processability, are mild to the skin, have improved smear and bar firmness properties, exhibit good lathering properties and/or reduced marring. The compositions of the present technology may also be utilized to produce dish washing pastes, gels and body washes, along with other uses. Additionally, the invention provides improved processes for manufacturing precursor cleansing/laundry bar “soap noodles,” personal cleansing bars and laundry detergent bars.

Particularly preferred embodiments presently disclosed comprise: between about 40% to about 94% by weight of a soap slurry, preferably comprised from tallow and/or coconut soap; from about 1% to about 15% by weight of a C₆-C₂₂ fatty acid; from about 1% to about 30% by weight of a mixture of (i) an alpha sulfonated alkyl ester, sulfonated fatty acid, or mixtures thereof; and (ii) a secondary synthetic surfactant, that is preferably an anionic, amphoteric, zwitterionic, nonionic, or semi-polar surfactant; between about 0.5% to about 2% by weight of an electrolyte that is preferably sodium sulfate, sodium chloride, sodium carbonate, potassium sulfate, potassium chloride, potassium carbonate, calcium sulfate, calcium chloride, calcium carbonate, calcium nitrate, magnesium sulfate, magnesium chloride, or magnesium carbonate, magnesium nitrate, mixtures thereof, derivatives thereof, alternatives thereof, or equivalents thereof; between about 0.5% to about 6.0% of a polyhydric alcohol; water; and optionally up to about 10% of an alkanolamide. Additionally, a substantial portion of one or more compositions of the presently described technology preferably exhibits or has a lamellar microstructure at about 70° C. while in slurry form.

Other embodiments of the present technology relate to an improved process to produce precursor cleansing/laundry bar “soap noodles,” and personal cleansing bars and/or laundry detergent bars derived from the soap bar compositions of the presently described technology. In a preferred embodiment, such a process comprises the steps of (a) forming at a temperature of about 65° C. to about 105° C. a substantially homogeneous aqueous liquid mixture comprising: an aqueous soap slurry comprising a C₆-C₂₂ soap, the slurry having a free alkalinity of less than about 0.1%; a C₆-C₂₂ fatty acid; an alpha sulfonated alkyl ester, a sulfonated fatty acid, or a mixture thereof; a secondary synthetic surfactant, that is preferably an anionic, amphoteric, zwitterionic, nonionic, or semi-polar surfactant; an electrolyte selected from sodium sulfate, sodium chloride, sodium carbonate, potassium sulfate, potassium chloride, potassium carbonate, calcium sulfate, calcium chloride, calcium carbonate, calcium nitrate, magnesium sulfate, magnesium chloride, magnesium carbonate, magnesium nitrate, derivatives thereof, and mixtures thereof; a polyhydric alcohol; and water in an amount from about 30% to about 36% by weight of the substantially homogeneous aqueous liquid mixture; wherein a substantial portion of the substantially homogeneous aqueous liquid mixture exhibits a lamellar microstructure at about 70° C.; and (b) drying the substantially homogeneous aqueous liquid mixture by removing water to form a thickened mixture comprising: from about 40% to about 94% by weight of the C₆-C₂₂ soap; from about 1% to about 15% by weight of the C₆-C₂₂ fatty acid; from about 2% to less than 12% by weight of the alpha sulfonated alkyl ester, the sulfonated fatty acid, or the mixture thereof; between about 0.5% to about 2% by weight of the electrolyte; between about 0.5% to about 6.0% by weight of the polyhydric alcohol; and between about 3% to about 22% by weight of water.

Processes of the present technology may include further steps, such as extruding the thickened mixture to form flaked solid or semi-solid particles, plodding the flaked solid or semi-solid particles to form plodded particles, and additional processing including a final extrusion step to form a billet. The final extrusion step is performed at a temperature from about 35° C. to about 45° C., and more preferably 35° C. to about 38° C. Once a billet has been formed, further processing steps may include, for example, cutting the billet to form a cut billet, and stamping the cut billet to yield a personal cleansing or a laundry detergent bar.

### DETAILED DESCRIPTION OF THE INVENTION

One embodiment of the present technology is a composition comprising: a soap, preferably tallow and/or coconut soap; primary surfactant comprising an alpha sulfonated alkyl ester, sulfonated fatty acid, and/or mixtures thereof; a C₆-C₂₂ fatty acid, an electrolyte (salt), a polyhydric alcohol, and water. Furthermore, embodiments preferably comprise one or more secondary synthetic surfactants. Examples of acceptable secondary synthetic surfactants include anionic, amphoteric, zwitterionic, nonionic, or semi-polar surfactants. Some embodiments of the present technology further include paraffin, and/or additional additives or surfactants. Optionally, the compositions may also contain alkanolamide.

Preferred embodiments of presently described compositions comprise: between about 40% to about 94% by weight of a soap, the soap is preferably tallow soap, coconut soap, or a mixture thereof; between about 1% to about 15% by weight of a C₆-C₂₂ fatty acid; from about 1% to about 30% by weight of a mixture of a primary suifactant and a secondary synthetic surfactant; between about 0.5% to about 2% of an electrolyte selected from the group consisting of sodium sulfate, sodium chloride, sodium carbonate, potassium sulfate, potassium chloride, potassium carbonate, calcium sulfate, calcium chloride, calcium carbonate, calcium nitrate, magnesium sulfate, magnesium chloride, magnesium carbonate, magnesium nitrate, derivatives thereof, mixtures thereof; alternatives thereof; and equivalents thereof; between about 0.5% to about 6% of a polyhydric alcohol; water; and optionally between about 0% to about 10% of an alkanolamide. The primary surfactant is preferably present in an amount less than 12% by weight of the composition, and is preferably an alpha sulfonated alkyl ester, a sulfonated fatty acid, or mixtures thereof. Additionally, a substantial portion of one or more soap bar compositions of the presently described technology preferably exhibit or have a lamellar microstructure at about 70° C. while in slurry form.

It should be understood that the specific amount of any component of the compositions of the present technology may be any value within the ranges described herein, depending upon the specific final composition make-up of components desired or utilized. For example, the soap component of the presently described technology can be present in any amount from about 40% to about 94% by weight of the soap bar composition; including any amount from about 40%, about 45%, about 50%, about 55%, about 60%, about 65%, or about 70% to about 94%, about 93%, about 92%, about 90%, about 85%, or about 80% and the like. The fatty acid component utilized in the soap bar compositions of the presently described technology can be present in any amount, including but not limited to, from about 1% to about 15%, including any amount from about 1%, about 2%, about 3%, or about 5%, to about 10%, about 8%, about 7%, or about 6%. The primary surfactant of the presently described technology can be present in any amount up to about less than 12% by weight of the composition, including but not limited to from about 2%, about 3%, about 3.5%, about 4%, or about 5%, to about 11%, about 10% or about 8%. The secondary surfactant component of the presently described technology can be present in any amount up to about 18% by weight of the composition, including but not limited to from about 1%, about 2%, about 3%, or about 5%, to about 17%, about 16%, about 15%, about 12% or about 10%. The electrolyte component of the presently described technology can be present in any amount, including but not limited to, from about 0.5%, about 0.7%, about 0.8%, or about 1%, to about 1.4%, about 1.6%, or about 1.8%. The polyhydric alcohol component of the presently described technology can be present in any amount, including but not limited to, from about 0.5%, 1%, or 2%, to about 4%, about 5%, or about 6%. Additional examples of component amounts that can be used in accordance with the present technology are provided in the discussion below.

Soap:

In accordance with this particular embodiment, the soap preferably has the following general chemical formula: 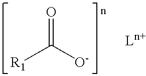
wherein R₁ is a C₆-C₂₂ hydrocarbyl group, an alkyl group, or combination thereof, n is 1 or 2, and L is a cation. Preferably, L. is sodium, potassium, calcium, magnesium, ammonium, monoethanolammonium, diethanolammonium, triethanolammonium, or a mixture thereof. Preferably, the soap is present as an aqueous slurry. The soap preferably comprises between about 40% to about 94% by weight of the initial mixture and/or thickened mixture, before or after drying or dehydration of the soap mixture. The soap can also be present in an amount from about 40% to about 92%, or from about 55% to about 94%, by weight of the soap bar composition. More preferably, the soap is present in an amount between about 65% to about 80% by weight of the composition. In some embodiments, the composition may comprise from about 56% to about 93% by weight of an aqueous soap slurry. It should be understood that the amount of soap put into a soap bar composition may vary depending upon the amount of other components to be added to the soap bar composition. The soap preferably comprises between about 65% to about 80% in a finished soap bar.

As stated above, the soap is preferably added to the initial soap bar composition in the form of an aqueous slurry. In a preferred embodiment, the aqueous slulTy is about 70% solids. The other components of the soap bar composition are mixed with the soap slurry to form an initial mixture. The primary source of the water content of the initial mixture is usually the water in this aqueous slurry, though additional water may be added if desired. Soap bar compositions of the present technology may have from about 3% to about 22%, more preferably from about 3% to about 16%, by weight of water at any point during processing. As part of the soap bar processing, most of the water is preferably removed from the initial mixture before forming a finished soap bar. Preferably, water comprises between about 3% to about 16% of a finished soap bar.

It is also preferable that, the soap is a tallow or coconut soap, or mixture thereof. Preferably, the soap comprises between about 60% to about 95% by weight of the soap of tallow soap and between about 5% to about 40% by weight of the soap of coconut soap. Most preferably, the soap comprises between about 60% to about 90% tallow soap and between about 10% to about 40% coconut soap.

Fatty Acid:

The fatty acid is preferably present from about 1% to about 15% by weight, and more preferably, between about 1% to about 7%. The fatty acid is preferably a C₆-C₂₂ fatty acid. The fatty acid preferably contains a hydrocarbyl group, an alkyl group, or combination thereof. More preferably, the fatty acid is a C₁₂-C₂₀ fatty acid. For example, in some preferred embodiments, the fatty acid has the formula: 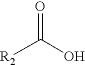
wherein R₂ is a C₆-C₂₂ hydrocarbyl group, an alkyl group, or a combination thereof. In a particularly preferred embodiment, R₂ is a C₁₂-C₂₀ hydrocarbyl group, or a combination of a C₁₂-C₂₀ hydrocarbyl group and an alkyl group.

The (free) fatty acids generally used in accordance with the present technology correspond with the fatty acids used to make conventional soaps. The fatty acid material which is desirably incorporated into the invention includes, for example, material ranging in hydrocarbon chain length of from about 6 to about 22 carbons, essentially saturated. These fatty acids can be highly purified individual chain lengths and/or crude mixtures such as those derived from fats and oils. The industry term “triple pressed stearic acid” comprises about 45 parts stearic and 55 parts palmitic acids. Additionally, the term stearic acid is used in the context of the soap industry to refer to a fatty acid mixture which is predominately stearic acid and shall be the meaning as used herein. Coconut fatty acids, and/or palm stearine fatty acids or combinations of thereof are also typically used as free fatty acid additives.

The composition and the methods of producing such compositions according to the present technology can include soaps derived from hydrocarbon chain lengths of from about 6 to about 22 carbons (including carboxyl carbon) and, in some embodiments, are saturated. In some manifestations of this particular embodiment described, the soap is the sodium salt, but other soluble soap can be used. Potassium, calcium, magnesium, monoethanolammonium, diethanolammonium, triethanolammonium, and mixtures thereof, are deemed acceptable. The soaps can be prepared by the in situ saponification or ion exchange with halide salt of the corresponding fatty acids, but they may also be introduced as pre-formed soaps.

Alpha Sulfonated Alkyl Ester or Alpha Sulfonated Fatty Acid:

The presently described compositions and processes preferably utilize an alpha sulfonated alkyl ester, alpha sulfonated fatty acid, or mixture thereof. The alpha sulfonated alkyl ester preferably has the following general formula: 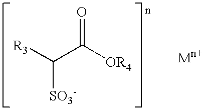
wherein R₃ is a C₆-C₂₂ hydrocarbyl group, an alkyl group, or combination thereof, R₄ is a straight or branched chain C₁-C₆ hydrocarbyl group, an alkyl group, or combination thereof, n is 1 or 2 and M is hydrogen, sodium, potassium, calcium, magnesium, ammonium, monoethanolammonium, diethanolammonium, triethanolammonium, a mixture thereof, a derivative thereof, an alternative thereof, or an equivalent thereof.

The sulfonated fatty acid preferably has the general formula: 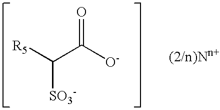
wherein R₅ is a C₆-C₂₂ hydrocarbyl group, an alkyl group, or combination thereof, n is 1 or 2 and wherein N is hydrogen, sodium, potassium, calcium, magnesium, ammonium, monoethanolammonium, diethanolammonium, triethanolammonium, a mixture thereof, a derivative thereof, an alternative thereof, or an equivalent thereof.

Embodiments of the present technology may disclose one or the other of such anionic surfactants, or a mixture of the two. Either a single such anionic surfactant or mixture of both types of anionic surfactants may also be utilized in combination with a secondary synthetic anionic, amphoteric, zwitterionic, nonionic, or semi-polar surfactant, as discussed below. In some embodiments alpha sulfonated alkyl esters and sulfonated fatty acids are present in a ratio of from about 0:1 to about 1:0. Some embodiments which utilize mixtures of alpha sulfonated alkyl esters and sulfonated fatty acids preferably utilize a ratio of from about 10:1 to about 1:10, or more preferably a ratio from about 3:1 to about 1:3.

The compositions of the presently described technology and the methods of producing such compositions preferably contain (or utilize) from about 1% to about 30% by weight of a suifactant mixture wherein the primary surfactant comprises an alpha sulfonated alkyl ester and/or sulfonated fatty acid. The primary surfactant is preferably present in an amount less than 12% by weight of the soap bar composition.

The alpha sulfonated alkyl esters used are typically prepared by sulfonating an alkyl ester of a fatty acid with a sulfonating agent such as SO₃, followed by neutralization with a base, such as sodium hydroxide, potassium hydroxide, calcium hydroxide, magnesium oxide, monoethanolamine, diethanolamine or triethanolamine, or a mixture thereof. When prepared in this manner, the alpha sulfonated alkyl esters normally contain a minor amount, typically not exceeding 33% by weight, of an alpha sulfonated fatty acid, i.e., di-salt, which results from hydrolysis of the ester. Generally, larger amounts of the di-salt are obtained by hydrolyzing a known amount of the monosalt; hydrolysis may be accomplished in situ during the preparation of the composition. Accordingly, the alpha sulfonated alkyl ester and alpha sulfonated fatty acid may be provided to the composition or utilized in the process of the presently described technology as a blend of components which naturally result from the sulfonation of an alkyl ester of a fatty acid, or as individual components. Furthermore, it is known to one skilled in the art that minor impurities such as sodium sulfate, unsulfonated methyl esters (ME), and unsulfonated fatty acids (FA) may also be present in the mixtures according to the present technology.

The alpha sulfonated alkyl esters, i.e., alkyl ester sulfonate surfactants, can include, for example, linear esters of C₆-C₂₂ carboxylic acid (i.e., fatty acids) which are sulfonated with gaseous SO₃ according to the “The Journal of American Oil Chemists Society,” 52 (1975), pp. 323-329. Suitable starting materials include, among others, natural fatty substances as derived from tallow, palm oil, etc. In some embodiments of the presently described technology, the α-sulfonated alkyl ester is a sulfonated methyl ester, desirably as further described herein.

Preferred embodiments, however, may contain either an alpha sulfonated alkyl ester separately, a sulfonated fatty acid separately, or a mixture of the two. Either component or a mixture of the components may be provided in any form, although preferably provided as an aqueous mixture (e.g., slurry).

Electrolyte (Salt):

Compositions and the methods of producing such compositions of the presently described technology generally contain (or utilize) about 0.5% to about 2%, or more preferably between about 0.8% to about 1.6%, by weight of an electrolyte. Without being bound by any particular theory, it is believed the electrolyte may be any salt capable of acting as crisping agent or builder to arrive at a final bar formulation. Preferably, the electrolyte is sodium sulfate, sodium chloride, sodium carbonate, potassium sulfate, potassium chloride, potassium carbonate, calcium sulfate, calcium chloride, calcium carbonate, calcium nitrate, magnesium sulfate, magnesium chloride, or magnesium carbonate, magnesium nitrate, mixtures thereof, derivatives thereof, alternatives thereof, or equivalents thereof. In a more preferred embodiment of the present technology the salt is magnesium chloride, sodium chloride or a mixture thereof. In a most preferred embodiment, the salt is sodium chloride.

Polyhydric Alcohol:

The polyhydric alcohol may be a polyol generally defined as a non-volatile di- or higher polyhydric alcohol, a sugar or a polyethylene glycol. In some preferred embodiments, the polyhydric alcohol is glycerin, polyglycerols, sorbitol, glycols, mixtures thereof, derivatives thereof, alternatives thereof, or equivalents thereof. Particular examples can include, without limitation, glycerine, propylene glycol, glycerol, sorbitol, sucrose and 200-400 molecular weight polyethylene glycol, dipropylene glycol, polypropylene glycols 2000, 4000, polyoxyethylene polyoxypropylene glycols, polyoxypropylene polyoxyethylene glycols, glycerol, sorbitol, ethoxylated sorbitol, hydroxypropyl sorbitol, polyethylene glycol 200-6000, methoxy polyethylene glycols 350, 550, 750, 2000, 5000, poly[ethylene oxide] homopolymers (100,000-5,000,000), polyalkylene glycols and derivatives, hexylene glycol (2-methyl-2,4-pentanediol), 1,3-butylene glycol, 1,2,6-hexanetriol, ethohexadiol USP (2-ethyl-1,3-hexanediol), C₁₅-C₁₈ vicinal glycol, and polyoxypropylene derivatives of trimethylolpropane.

The useful polyols of the present technology are generally liquid water-soluble aliphatic polyols or polyethylene glycols or polypropylene glycols. The polyol may be saturated or contain ethylenic linkages; it must have at least two alcohol groups attached to separate carbon atoms in the chain, and must be water soluble and liquid at room temperature. If desired, the compound may have an alcohol group attached to each carbon atom in the chain. Among the compounds which are effective are, for example, ethylene glycol, propylene glycol, glycerine and mixtures thereof. In some embodiments, the polyol is glycerine. Water-soluble polyethylene glycols, water-soluble polypropylene glycols useful in accordance with the technology of the present invention are those products produced by the condensation of ethylene glycol molecules or propylene glycol molecules to form high molecular weight ethers having terminal hydroxyl groups. The polyethylene glycol compounds may range from diethylene glycol to those having molecular weights as high as about 800, and, in some embodiments, about 100 to 700, in other embodiments, 100 to 600. Normally, polyethylene glycols having molecular weights up to 800 are liquid and completely soluble in water. As the molecular weight of the polyethylene glycol increases beyond 800, they become solid and less water-soluble. Such solids may be used as plasticizers herein when malleable at 35° C. to about 46° C. The polypropylene glycol compounds may range from dipropylene glycol to polypropylene glycols having molecular weights of about 2000, and, in some embodiments, less than 1500, in other embodiments, less than 1000. These are normally liquid at room temperature and are readily soluble in water.

Secondary Synthetic Surfactant:

The present technology also preferably comprises a secondary synthetic surfactant in combination with the alpha sulfonated alkyl ester, sulfonated fatty acid, or mixtures thereof. Preferably, the secondary synthetic surfactant is present in an amount such that the mixture of total surfactant is between about 1% to about 30% by weight of the total composition. More preferably, the secondary synthetic surfactant is present in an amount between about 5% to about 15% by weight of the total composition.

Secondary synthetic suifactants are preferably anionic, amphoteric, zwitterionic, nonionic, or semi-polar surfactants. Preferred synthetic surfactants include, for example, alkylamidopropyl betaine, alkylamidopropyl hydroxysultaine, a salt of alkylamphoacetate, a salt of alkyl sulfoacetate, a di-salt of alkyethoxy sulfosuccinate, a di-salt of alkyl sulfosuccinate, alkylamide monoethanolamine, alkylamidopropylamine oxide, a salt of alpha olefin sulfonate, a salt of alkyl sulfate, a salt of alkylyl isethionate, a salt of alkylethoxy sulfate, a salt of alkyliminodipropionate, a salt of alkyl sarcosinate, a salt of alkyethoxy sarcosinate, alkylpolyglycoside, a salt of alkyl lactylate, a salt of ethoxylated alkyl lactylate, a salt of alkenyl lactylte, a salt of ethoxylated alkenyl actylate, a salt of alkyl amphoacetate, combinations thereof, derivatives thereof, alternatives thereof, and equivalents thereof.

Contemplated secondary synthetic surfactants further include, but are not limited to the following: cocoamidopropyl betaine, laurylamidopropyl betaine, cocoamidopropyl hydroxysultaine, sodium cocoamphoacetate, sodium lauryl sulfoacetate, sodium laureth sulfoacetate, disodium laureth sulfosuccinate, disodium lauryl sulfosuccinate, cocoamide monoethanolamine, cocoamidopropylamine oxide, laurylamidopropylamine oxide, lauryl/myristylamidopropylamine oxide, sodium alpha olefin sulfonate, sodium lauryl sulfate, sodium cocoyl isethionate, sodium lauryl ether sulfate, potassium lauryl sulfate, magnesium lauryl sulfate, sodium lauriminodipropionate, sodium lauryl sarcosinate, sodium laureth sarcosinate, alkylpolyglycoside, sodium lauryl lactylate, sodium ethoxylated lauryl lactylate, sodium lauryl amphoacetate, sodium coco sulfate, mixtures thereof, and derivatives thereof.

More preferably, the secondary synthetic surfactant is cocoamidopropyl betaine, sodium lauryl sulfoacetate, disodium laureth sulfosuccinate, acyl lactylate, sodium alpha olefin sulfonate, potassium lauryl sulfate, sodium coco sulfate or sodium laureth sulfate. Most preferably, the secondary synthetic surfactant is cocoamidopropyl betaine.

Additional Ingredients:

The presently disclosed compositions may optionally further comprise an alkanolamide having the following general formula: 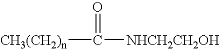
wherein n=6-16. Preferably, the alkanolamide is present in an amount up to about 10% by weight of the composition, more preferably between about 1% to about 10%, and most preferably between about 2% to about 5%.

The compositions and the methods of producing such compositions also optionally may further comprise (or utilize) additional ingredients, surfactants, pH adjusters, emollients, moisturizers, viscocity agents, buffers, and the like as disclosed in published PCT Application WO 03/063819, to Ospinal et al., published on Aug. 7, 2003, incorporated by reference herein.

For example, some additives may include from about 0.5% to about 10% by weight of a sucrogylceride, a functional metallic soap, a succinamate, a sulfosuccinamate, a mono-, di-, or trigylceride, chitosan, or a mixture thereof. Similarly, the compositions and the methods of producing such compositions may further comprise (or utilize) from about 0.1% to about 10% by weight of fragrance, emollients, moisturizers, viscosity control agents, as well as additional agents appropriate for incorporation into a composition of the invention and which are known to those skilled in the art.

Other optional additives may include additional detergent surfactants, such as for example, acyl isethionates, e.g., sodium acyl (cocoyl) isethionate (SCI). Examples of suitable anionic surfactants include, among others, the sodium, potassium, magnesium, calcium, ammonium, monoethanolammonium (MEA), diethanolammonium (DEA), triethanolammonium (TEA), or alkyl amine salts, or mixtures thereof, of sulfonic acids, polysulfonic acids, sulfonic acids of oils, paraffin sulfonic acids, lignin sulfonic acids, petroleum sulfonic acids, tall oil acids, olefin sulfonic acids, hydroxyolefin sulfonic acids, polyolefin sulfonic acids, polyhydroxy polyolefin sulfonic acids, perfluorinated carboxylic acids, alkoxylated carboxylic acid sulfonic acids, polycarboxylic acids, polycarboxylic acid polysulfonic acids, alkoxylated polycarboxylic acid polysulfonic acids, phosphoric acids, alkoxylated phosphoric acids, polyphosphoric acids, and alkoxylated polyphosphoric acids, fluorinated phosphoric acids, phosphoric acid esters of oils, phosphinic acids, alkylphosphinic acids, aminophosphinic acids, polyphosphinic acids, vinyl phosphinic acids, phosphonic acids, polyphosphonic acids, phosphonic acid alkyl esters, α-phosphono fatty acids, oragnoamine polymethylphosphonic acids, organoamino dialkylene phosphonic acids, alkanolamine phosphonic acids, trialkyledine phosphonic acids, acylamidomethane phosphonic acids, alkyliminodimethylene diphosphonic acids, polymethylene-bis(nitrilo dimethylene)tetraphosphonic acids, alkyl bis(phosphonoalkylidene) amine oxide acids, esters of substituted aminomethylphosphonic acids, phosphonamidic acids, acylated amino acids (e.g., amino acids reacted with alkyl acyl chlorides, alkyl esters or carboxylic acids to produce N-acylamino acids), N-alkyl acylamino acids, acylated protein hydrolysates, branched alkylbenzene sulfonic acids, alkyl gylceryl ether sulfuric acid esters, alkyl sulfuric acid esters, alkoxylated alkyl sulfuric acid esters, α-sulfonated ester diacids, alkoxylated α-sulfonated alkyl ester acids, α-sulfonated dialkyl diester acids, di-α-sulfonated dialkyl diester acids, α-sulfonated alkyl acetate acids, primary and secondary alkyl sulfonic acids, perfluorinated alkyl sulfonic acids, sulfosuccinic mono- and diester acids, polysulfosuccinic polyester acids, sulfoitaconic diester acids, sulfosuccinamic acids, sulfosuccinic amide acids, sulfosuccinic imide acids, phthalic acids, sulfophthalic acids, sulfoisophthalic acids, phthalamic acids, sulfophthalamic acids, alkyl ketone sulfonic acids, hydroxyalkane-1-sulfonic acids, lactone sulfonic acids, sulfonic acid amides, sulfonic acid diamides, alkyl phenol sulfuric acid esters, alkoxylated alkyl phenol sulfuric acid esters, alkylated cycloalkyl sulfuric acid esters, alkoxylated alkylated cycloalkyl sulfuric acid esters, dendritic polysulfonic acids, dendritic polycarboxylic acids, dendritic polyphosphoric acids, sarcosinic acids, isethionic acids, tauric acids, fluorinated carboxylic acids, fluorinated sulfonic acids, fluorinated sulfate acids, fluorinated phosphonic and phosphinic acids, mixtures thereof, derivatives thereof, alternatives thereof, and equivalents thereof.

Suitable nonionic surfactants include those generally disclosed in U.S. Pat. No. 3,929,678, Laughlin et al., issued on Dec. 30, 1975, at column, 13 line 14 through column 16, line 6, incorporated herein by reference. Other suitable nonionic surfactants may include, for example, those selected from the group comprising polyoxyethyleneated alkylphenols, polyoxyethyleneated straight chain alcohols, polyoxyethyleneated branched chain alcohols, polyoxyethyleneated polyoxypropylene glycols, polyoxyethyleneated mercaptans, fatty acid esters, glyceryl fatty acid esters, polyglyceryl fatty acid esters, propylene glycol esters, sorbitol esters, polyoxyethyleneated sorbitol esters, polyoxyethylene glycol esters, polyoxyethyleneated fatty acid esters, primary alkanolamides, ethoxylated primary alkanolamides, secondary alkanolamides, ethoxylated secondary alkanolamides, tertiary acetylenic glycols, polyoxyethyleneated silicones, N-alkylpyrrolidones, alkylpolyglycosides, alkylpolylsaccharides, EO-PO block polymers, polyhydroxy fatty acid amides, amine oxides, mixtures thereof, derivatives thereof, alternatives thereof, and equivalents thereof.

The compositions and the methods of producing such compositions herein may be formulated and carried out such that they will have a pH of between about 4.0 and about 10.0, and, in some embodiments, between about 5 and about 9.5. Techniques for controlling pH at recommended usage levels include the use of buffers, alkali, acids, etc., and are well known to those skilled in the art. Optional pH adjusting agents can include, but are not limited to citric acid, succinic acid, phosphoric acid, sodium hydroxide, sodium carbonate, and the like.

Other optional ingredients can include sequestering agents such as disodium ethylenediamine tetraacetate, auxiliary surfactants are selected from the group comprising amides, amine oxides, betaines, sultaines and C₈-C₁₈ fatty alcohols, hydrating cationic polymer, suitable plasticizers, non-volatile, nonionic silicone conditioning agents, polyalkyl or polyaryl siloxanes, and pearlescent/suspending agents, detergent builders, anti-bacterial agents, fluorescers, dyes or pigments, polymers, perfumes, cellulase enzymes, softening clays, smectite-type softening clays, polymeric clays, flocculating agents, dye transfer inhibitors, optical brighteners, skin feel enhancers including aluminosilicate and non-aluminosilicate odor-controlling materials, chitan, triglycerides, glycerine, succinamates, sucroglycerides, functional metallo-soaps, and mixtures thereof.

The compositions of the presently described technology may be transparent and/or produce a transparent personal cleansing or laundry detergent bar upon proper processing and/or selection of optional ingredients and components detailed herein. Additionally, the compositions may be used to produce a transparent dish washing gel, paste or solution, or further applications or forms which will be apparent to one skilled in the art. Whether transparent or nontransparent, the compositions may exist as solid flakes, or as a gel.

Further, the compositions and the methods of producing such compositions of the present technology may optionally contain (or utilize) about 1.0% to about 15.0% by weight of a wax, in some embodiments, for example, paraffin, having a melting point of from about 54° C. to about 180° C. The waxes can include without limitation beeswax, spermaceti, carnauba, bayberry, candelilla, montan, ozokerite, ceresin, paraffin, synthetic waxes such as Fisher-Tropsch waxes, microcrystalline wax, derivatives thereof, or mixtures thereof. The wax ingredient is used in the compositions of the present technology to impart skin mildness, plasticity, firmness, and processability. Wax also provides a glossy look and smooth feel to the final product.

Thus, at least one additional component of the compositions of the present technology can be a wax, and in some embodiments, paraffin wax having a melting point of from about 54° C. to about 82° C., in other embodiments from about 60° C. to about 74° C., and in yet other embodiments from about 61° C. to about 71° C. “High melt” paraffin is a paraffin that has a melting point from about 66° C. to about 71° C. “Low melt” paraffin is a paraffin that has a melting point from about 54° C. to about 60° C. In some embodiments, the paraffin wax is a fully refined petroleum wax which is odorless and tasteless and meets FDA requirements for use as coatings for food and food packages. Such paraffins are readily available commercially. A suitable paraffin can be obtained, for example, from The National Wax Co. under the trade name 6975.

Processing:

Other embodiments of the present technology relate to an improved process to produce precursor cleansing/laundry bar “soap noodles,” personal cleansing bars and laundry detergent bars derived from the compositions presently described.

Such a process preferably comprises first forming at a temperature of about 65° C. to about 105° C. a substantially homogeneous aqueous liquid mixture comprising: an aqueous soap slurry comprising a C₆-C₂₂ soap; a C₆-C₂₂ fatty acid; a surfactant mixture; an electrolyte selected from the group consisting of sodium sulfate, sodium chloride, sodium carbonate, potassium sulfate, potassium chloride, potassium carbonate, calcium sulfate, calcium chloride, calcium carbonate, calcium nitrate, magnesium sulfate, magnesium chloride, magnesium carbonate, magnesium nitrate, mixtures thereof, derivatives thereof, alternatives thereof, and equivalents thereof; a polyhydric alcohol; and water. Preferred surfactant mixtures comprise a mixture of either a sulfonated fatty acid or an alpha sulfonated alkyl ester, plus a secondary synthetic surfactant. Most preferably, both sulfonated fatty acid and alpha sulfonated alkyl ester are utilized in combination with a secondary synthetic surfactant. In at least one preferred embodiment, the aqueous soap slurry comprising a C₆-C₂₂ soap preferably has a free alkalinity of less than about 0.1%. It is also preferred that the water content be in an amount from about 30% to about 36% by weight of the substantially homogeneous aqueous liquid mixture. Additionally, it is preferred that a substantial portion of the substantially homogeneous aqueous liquid mixture exhibits a lamellar microstructure at about 70° C.

Next, the process preferably involves drying the substantially homogeneous aqueous liquid mixture by removing water to form a thickened mixture. The thickened mixture may comprise amounts of the components of the homogeneous aqueous liquid mixture in any amount in accordance with the soap bar compositions described above. In some preferred embodiments, for example, the thickened mixture comprises from about 40% to about 94%, more preferably from about 60% to about 75%, by weight of the C₆-C₂₂ soap; from about 1% to about 15%, more preferably from about 1% to about 7%, by weight of the C₆-C₂₂ fatty acid; from about 1% to about 30% by weight of a mixture of surfactants; between about 0.5% to about 2% by weight of the electrolyte; between about 0.5% to about 6.0%, more preferably between about 1% to about 4%, by weight of the polyhydric alcohol; and between about 3% to about 22%, more preferably between about 3% to about 16%, and most preferably between about 9% and about 12%, by weight of water. Preferred surfactant mixtures comprise an alpha sulfonated alkyl ester, a sulfonated fatty acid, or a mixture thereof in an amount from about 2%, more preferably from about 5%, to less than 12% by weight of the overall composition, and a secondary synthetic surfactant.

Removal of the water from the initial liquid mixture is preferably accomplished by scraped wall vacuum evaporation drying under reduced pressure or heated drum drying at ambient pressure. In a preferred embodiment, about 55% to about 85% by weight of the water is removed from the initial liquid mixture; and most preferably, about 60% to about 80% by weight of the water is removed from the initial liquid mixture. Some examples of determining water removal by drying include final thickened mixtures comprising between about 1.74% of the final thickened mixture (Example: approximately 70% solids of an aqueous sluny comprising 58% of the initial mixture, with 90% water removed) to about 26.5% of the final mixture (Example: approximately 70% solids of an aqueous slurry comprising 93% of the initial mixture, with 5% water removed). Other examples of determining water removal by drying include final thickened mixtures comprising between about 3.48% of the final thickened mixture (Example: approximately 70% solids of an aqueous slurry comprising 58% of the initial mixture, with 80% water removed) to about 11.16% of the final mixture (Example: approximately 70% solids of an aqueous slurry comprising 93% of the initial mixture, with 60% removed).

Processes of the present technology may include further steps, such as extruding the thickened mixture to form flaked solid or semi-solid particles, plodding the flaked solid or semi-solid particles to form plodded particles, and additional processing including a final extrusion step to form a billet. The final extrusion step is perfonmed at a temperature from about 35° C. to about 45° C., and more preferably 35° C. to about 38° C. Once a billet has been formed, further processing steps may include, for example, cutting the billet to form a cut billet, and stamping the cut billet to yield a personal cleansing or a laundry detergent bar.

The processes of the present technology described herein generally overcome many of the shortcomings of the aforementioned heretofore known processes. For example, the present technology yields substantially homogeneous soap noodles which results in bars with minimal glit or hard specks. The processes are also carried out at temperatures at or below about 105° C. in the atmospheric mixing stage (i.e., forming the homogeneous aqueous liquid mixture) so as to conserve energy and minimize hydrolysis of the alpha sulfonated alkyl ester, and the process utilizes standard bar processing equipment. Furthermore, soap bars resulting from the improved process have the desired hardness, water permeability, low grit, enhanced slip, reduced hard specks, and an absence of marring (even when dried to exceptionally low moisture levels, and with aging on the shelf for several months).

While compositions of the present technology are extremely useful in soap bar and laundry bar applications, other applications for these compositions are possible. The compositions of the presently described technology may be useable in or as liquid, paste or gel dish washing compositions, hand soaps including waterless hand cleaners, multi-purpose cleaners, body washes, further laundry detergent compositions such as laundry powder, pre-spotter or stain sticks, textile treatment compositions including triethanolamine (TEA) soaps for dry cleaning, shampoos including those for humans, pets, and carpets, car wash, soap scouring pads and scrubbing pads, toilet tank drop ins and/or cleaners, personal care creams and lotions, and the like.

Definitions, Abbreviations, and CTFA Designations

The definitions, abbreviations, and CTFA designations used in the invention are as set forth as follows:
BHT butylated hydroxytoluene (di-tert-butyl-p-cresol)
BHA butylated hydroxyanisole (3-t-butyl-4-hydroxyanisole)
Coco Fatty Acid Emery 627 (a tradename from Emery Corporation, a division of Henkel) and coconut fatty acids that can be substituted for Emery 627
EDTA ethylenediamine tetraacetic acid, available from Dow Chemical Company.
Hyamine di-isobutyl-phenoxy-ethoxy-ethyl-dimethyl-benzyl ammonium chloride
ALPHA-STEP® MC-48 average about 5:1 to about 10:1 mixture of sulfonated stripped coco methyl esters and stripped coco fatty acids available from Stepan Company
NINOL® CMP or LMP CMP—coco monethanolamine amide, available from Stepan Company LMP—lauric/myristic (C12-C14 monethanolamine amide), available from Stepan Company
Pristerene 4981 Stearic Acid (from Unichema); approximate iodine value of 1.0 maximum; mixture of about 65% C₁₈ fatty acid, about 28% C₁₆ fatty acid and about 2% myristic fatty acid
SFA disalt; α-sulfonated fatty acid (e.g., resulting from hydrolysis of SME)
SME monosalt; α-sulfonated alkyl ester (e.g., α-sulfonated methyl ester)
UA unreacted methyl ester
ALPHA STEP® BSS-45 average about 1.3 to about 1.8:1 mixture of alpha sulfonated stripped coco methyl esters and stripped coco fatty acids with actives of about 43% to about 45% available from Stepan Company
ALPHA-STEP® PC-48 coco 10:1 SME to SFA ratio, available from Stepan Company
ALPHA-STEP® PS-65 palm stearin 10:1 SME to SFA ratio, available from Stepan Company
ALPHA-STEP® PS-45 SF palm stearin 1.5:1 SME to SFA ratio, available from Stepan Company
LATHANOL® sodium lauryl sulfoacetate, available from Stepan Company LAL Powder
AMMONYX® HCDO cocoamidopropylamine oxide, available from Stepan Company
BIO-TERGE® AS-40 HA Na C₁₄-C₁₆ olefin sulfonate, available from Stepan Company
STEOL® CS-370 sodium laureth sulfate, available from Stepan Company
STEPANOL® P-30 potassium lauryl sulfate, available from Stepan Company
Stepanol® MG magnesium lauryl sulfate, available from Stepan Company
NINOL® C-5 PCG-6 cocamide, available from Stepan Company
MAPROSYL® 30 sodium lauryl/sarcosinate, available from Stepan Company
ALPHA-STEP® DS-85 palm stearin 100% SFA, available from Stepan Company
ALPHA-STEP® BSS-85 coco 100% SFA, available from Stepan Company
AMPHOSOL® HCG cocoamidopropyl betaine, available from Stepan Company

The present technology is illustrated in the following non-limiting Examples. All proportions in the examples and elsewhere in the specification are by weight unless specifically stated otherwise.

All documents, e.g., patents and journal articles, cited above or below are hereby incorporated by reference in their entirety. One skilled in the art will recognize that modifications may be made in the invention without deviating from the spirit or scope of the invention. The invention is illustrated further by the following examples which are not to be construed as limiting the invention or scope of the specific procedures or compositions described herein. All levels and ranges, temperatures, results etc., used herein are approximations unless otherwise specified.

### EXAMPLES

### Example 1

### Procedure for Making Cleaning Bar

One procedure for making soap/SME and/or SFA combo bars is as follows:
(1) Neat soap is melted in a steam jacketed crutcher (about 140° F. to about 200° F.)
(2) Free alkalinity of neat soap is neutralized to about 0.1% maximum with inorganic acids, such as phosphoric acid, or organic acid such us coco fatty acids, or citric acid.
(3 ) Alpha sulfomethyl ester, alpha sulfonated fatty acid or mixtures thereof, as a dried paste or an aqueous solution, is added to the crutcher with stirring, and agitation is continued for about 5 minutes.
(4 ) Additives, such as stearic acid and/or coco fatty acids, mixtures thereof (about 1 to about 5%) glycerine (about 0.5% to about 4.0%) and sodium chloride (about 0.1% to about 2.0%) can be introduced into the crutcher at this point and stirring continued for about another 2 to 5 minutes.
(5) The wet soap is air-dried or vacuum-dried to reduce the moisture level to below about 5%.
(6 ) To milled soap chips, perfume, titanium dioxide and other minor additives are added and milled again (this time with the crimper plate in position).
(7 ) The soap mix is processed through a Mazzoni plodder, commercially available from Stephan Beck Plodder Co. The temperature of the plodder is maintained at about 90° F. to about 100° F. using a water circulation system.
(8 ) Bars are pressed from the extruded ribbon using a Midget Multipress (commercially available from Denison Co. equipped with a standard rectangular die.

### Example 2

### Di-Salt Sulfonated Fatty Acid (SFA) Preparation

Approximately 3500 grams of MC-48 acid is placed in a 4 L beaker and with rapid agitation, approximately 330 grams of sodium hydroxide is added slowly. Upon complete addition of the sodium hydroxide, the resulting SFA material had a thick, pasty consistency. The crude SFA is re-crystallized by washing with methanol, water and salting out the purified SFA product. The crude SFA is analyzed by titrating the material with 0.02N hyamine, which indicated that approximately 46.6% di-sodium salt is present in MC-48 is present. The recrystallized SFA product is approximately 99.8% di-sodium salt.

### Example 3:1:1

### Ratio of SME to SFA Sample Preparation

Approximately 138.5 grams of MC-48 acid is added to a 1 L resin kettle, equipped with heating means, agitation means, pH measurement means and a nitrogen sweep. The acid is heated to about 55° C. and approximately 18.7 g of sodium hydroxide powder is added in small portions. As the sodium hydroxide is added an exotherm of about 55° C. to about 71° C. occurred, during which time cooling is provided to keep the mixture below approximately 80° C. Near the end of the sodium hydroxide addition, the mixture became very thick and approximately 15.6 grams of methanol is added to keep the mixture semi-fluid. The final product is a paste at room temperature, i.e. about 25° C. The final SFA/SME product is titrated with 0.02N hyamine which showed the material to be approximately 41.65% SME (mono salt) and approximately 40.34% SFA (di-salt).

### Example 4

### 2:1 Ratio SME to SFA Sample Preparation

Approximately 53.4 grams of undigested α-sulfomethyl ester acid is placed in a 500 mL 4-neck flask, equipped with a heating means, a condenser and stirring means. The acid is heated to about 130° C. for about 1 minute to digest the acid. The acid is cooled after digestion to about 75° C., and approximately 5.3 grams of anhydrous methanol is added, which produced an exotherm to approximately 85° C. Next, approximately 6.4 grams hydrogen peroxide (35% solution) is added and the resulting mixture heated to about 120° C. for about 5 minutes. After this period of time, the mixture is cooled to about 60° C. and approximately 8.82 grams water is added, producing a gel-like mixture. The mixture is then further cooled to about 40° C. and sodium hydroxide (50% solution.) is added dropwise until a pH of 6 is achieved. The final product is a soft, flowable, yellow gel. The actives are determined, via titration with 0.02N hyamine, to be 46.3% SME (mono-salt) and 22.5 SFA (di-salt).

### Example 5

### 25:1 Ratio SME to SFA Sample Preparation

Approximately 50 grams of undigested α-sulfomethyl ester acid is placed in a 500 mL round bottom flask and heated to about 130° C. for about 1 minute using a hot oil bath. A mechanical stirrer with a glass shaft and teflon blade is used to ensure thorough mixing. The apparatus included a condenser (allihn type) to prevent loss of any solvent vapors. The acid is cooled after digestion to about 70° C., and approximately 5.3 grams of anhydrous methanol is added and thoroughly combined. This is followed by the addition of approximately 1.825 grams hydrogen peroxide (50% solution.) and heating of the resulting mixture to about 89° C. for about 64 minutes. After this period of time, the mixture is cooled to about 40° C. and approximately 64.7 grams water is added and mixed thoroughly. The acid is neutralized by the dropwise addition of sodium hydroxide (50% solution) until a pH of about 6.5 is achieved, all the while maintaining the temperature below about 45° C. using a water/ice bath. The final product is analyzed by titration with 0.02N hyamine, and found to comprise 35.82% SME (mono-salt) and 1.36 SFA (di-salt), with the SME:SFA ratio being 26.3:1.

### Example 6

### Preparation of Samples Containing Various Amounts of SME and SFA

In general, samples containing differing amounts of SFA and SME (e.g., total amounts of each or either present in the initial liquid mixture, and optionally present with respect to varying amounts of total SFA and SME actives) can be obtained, for instance, by varying the hydrolysis of SME to SFA (e.g., by varying hydrolysis conditions, and/or amount of methanol applied for hydrolysis). Similarly, mixtures can be combined, and/or varying amounts of either pure (or relatively pure) SME or SFA can be added to adjust the concentration of a particular mixture. One skilled in the art will recognize how to obtain the particular ratios referenced herein (if not otherwise disclosed) as well as further ratios and formulations encompassed by the scope of the presently described technology and appended claims.

### Example 7

### Cleaning Bar Formulations

Table 1 provides two soap bar formulations without alpha sulfonated alkyl ester or sulfonated fatty acid (Formulation A), or without polyhydric alcohol (Formulation B), used herein as control formulations. An additional control formulation is provided in Table 7. Tables 2-7 provide examples of formulations of skin cleansing bars according to the present technology, indicating weight percent of components in finished cleansing bars.

TABLE 1

| | Control | Control |
|---|---|---|
| | Formulation A | Formulation B |
| Components | (Weight % Active) | (Weight % Active) |
| Tallow/coco soap | 81.3 | 69.8 |
| (80/20) | | |
| ALPHA -STEP ® BSS- | 0.0 | 15.0 |
| 45 | | |
| Coconut Fatty Acids | 4.0 | 4.0 |
| Glycerin | 3.5 | 0.0 |
| Sodium Chloride | 1.0 | 1.0 |
| Water | 10.0 | 10.0 |
| Minor additives | 0.2 | 0.2 |
| (Citric Acid, EDTA) | | |
| TOTAL | 100.0 | 100.0 |

TABLE 2

| | Formulation 1 | Formulation 2 | Formulation 3 | Formulation 4 |
|---|---|---|---|---|
| Components | (Wt. % Active) | (Wt. % Active) | (Wt. % Active) | (Wt. % Active) |
| Tallow/coco soap (85/15) | 75.8 | 69.8 | 67.8 | 63.9 |
| ALPHA-STEP ® BSS- 45 | 7.5 | 7.5 | 7.5 | 15.0 |
| Coconut Fatty Acids | 1.0 | 6.0 | 8.0 | 2.0 |
| Glycerin | 1.0 | 2.0 | 2.0 | 3.5 |
| Sodium Chloride | 0.5 | 0.5 | 0.5 | 1.4 |
| Water | 10.0 | 10.0 | 10.0 | 10.0 |
| Fragrance | 1.2 | 1.2 | 1.2 | 1.2 |
| Minor additives | 3.0 | 3.0 | 3.0 | 3.0 |
| (colorants, antioxidants, | | | | |
| EDTA, fillers, etc.) | | | | |
| TOTAL | 100.0 | 100.0 | 100.0 | 100.0 |

TABLE 3

| | Formulation 5 | Formulation 6 | Formulation 7 | Formulation 8 |
|---|---|---|---|---|
| Components | (Wt. % Active) | (Wt. % Active) | (Wt. % Active) | (Wt. % Active) |
| Tallow/coco soap | 61.9 | 60.3 | 52.8 | 50.8 |
| (85/15) | | | | |
| ALPHA-STEP ® | 15.0 | 15.0 | 15.0 | 20.0 |
| BSS-45 | | | | |
| Coconut Fatty | 4.0 | 6.0 | 10.0 | 10.0 |
| Acids | | | | |
| Glycerin | 3.5 | 3.5 | 7.0 | 4.0 |
| Sodium Chloride | 1.4 | 1.0 | 1.0 | 1.0 |
| Water | 10.0 | 10.0 | 10.0 | 10.0 |
| Fragrance | 1.2 | 1.2 | 1.2 | 1.2 |
| Minor additives | 3.0 | 3.0 | 3.0 | 3.0 |
| (colorants, | | | | |
| antioxidants, | | | | |
| EDTA, fillers, etc.) | | | | |
| TOTAL | 100.0 | 100.0 | 100.0 | 100.0 |

TABLE 4

| | Formulation 9 | Formulation 10 | Formulation 11 | Formulation 12 |
|---|---|---|---|---|
| Components | (Wt. % Active) | (Wt. % Active) | (Wt. % Active) | (Wt. % Active) |
| Tallow/coco soap | 60.3 | 60.3 | 60.3 | 60.3 |
| (85/15) | | | | |
| ALPHA-STEP ® BSS- | 12.0 | 12.0 | 10.0 | 10.0 |
| 45 | | | | |
| NINOL ® CMP or LMP | 3.0¹ | 3.0² | 5.0¹ | 5.0² |
| Stearic/Coconut Fatty | 6.0 | 6.0 | 6.0 | 6.0 |
| Acids (85:15) | | | | |
| Glycerin | 3.5 | 3.5 | 3.5 | 3.5 |
| Sodium Chloride | 1.0 | 1.0 | 1.0 | 1.0 |
| Water | 10.0 | 10.0 | 10.0 | 10.0 |
| Fragrance | 1.2 | 1.2 | 1.2 | 1.2 |
| Minor additives | 3.0 | 3.0 | 3.0 | 3.0 |
| (colorants, antioxidants, | | | | |
| EDTA, fillers, etc.) | | | | |
| TOTAL | 100.0 | 100.0 | 100.0 | 100.0 |

Note
¹NINOL ® LMP (LMP: Lauryl Monoethanolamide)
Note
²NINOL ® CMP (CMP: Coconut Monoethanolamide)

TABLE 5

| | Formulation 13 | Formulation 14A | Formulation 14B |
|---|---|---|---|
| Components | Wt. % Active | Wt. % Active | Wt. % Active |
| Tallow/coco soap (85/15) | 55.3 | 55.3 | 65.3 |
| ALPHA-STEP ® BSS-45 | 15.0 | 15.0 | 6.7 |
| NINOL ® CMP or LMP | 5.0¹ | 5.0² | 3.3 |
| Coconut Fatty Acids | 6.0 | 6.0 | 6.0 |
| Glycerin | 3.5 | 3.5 | 3.5 |
| Salt | 1.0³ | 1.0³ | 1.0⁴ |
| Water | 10.0 | 10.0 | 10.0 |
| Fragrance | 1.2 | 1.2 | 1.2 |
| Minor additives (colorants, | 3.0 | 3.0 | 3.0 |
| antioxidants, EDTA, fillers, | | | |
| etc.) | | | |
| TOTAL | 100.0 | 100.0 | 100.0 |

Note
¹NINOL ® LMP
Note
²NINOL ® CMP
Note
³Salt is sodium chloride
Note
⁴Salt is 1:1 sodium chloride:magnesium sulfate

TABLE 6

| | Formulation 15 | Formulation 16 |
|---|---|---|
| Components | Wt. % Active | Wt. % Active |
| Tallow/coco soap (80/20) | 66.3 | 64.8 |
| ALPHA-STEP ® BSS-45 | 15.0 | 15.0 |
| Coconut Fatty Acids | 4.0 | 4.0 |
| Glycerin | 3.5 | 5.0 |
| Sodium Chloride | 1.0 | 1.0 |
| Water | 10.0 | 10.0 |
| Minor additives (colorants, | 0.2 | 0.2 |
| antioxidants, EDTA, fillers, | | |
| etc.) | | |
| TOTAL | 100.0 | 100.0 |

TABLE 7

| | Formulation C | | |
|---|---|---|---|
| | (Control) | Formulation 17 | Formulation 18 |
| Components | (Wt. % Active) | (Wt. % Active) | (Wt. % Active) |
| Sodium Tallow/Coco Soap | 82.5 | 72.93 | 68.14 |
| (80/20) | | | |
| ALPHA-STEP ® BSS-45 | 0 | 8 | 12 |
| Sodium Chloride | 1 | 1 | 1 |
| Glycerin | 3.5 | 3.5 | 3.5 |
| Stearic/Coconut Fatty | 4 | 4 | 4 |
| Acids (1:1) | | | |
| Water | 9 | 9 | 9 |
| Inactives | 0 | 1.57 | 2.36 |
| Additives (Fragrance, | 0 | 0 | 0 |
| Titanium, etc.) | | | |
| TOTAL | 100.0 | 100.0 | 100.0 |

### Example 8

### Formulations of Cleaning Bars with Additional Synthetic Secondary Surfactant

Tables 8-18 provide examples of formulations of skin cleansing bars with added secondary synthetic surfactant, indicating weight percent of components in finished cleansing bars.

TABLE 8

| | Formulation 19 | Formulation 20 | Formulation 21 | Formulation 22 |
|---|---|---|---|---|
| Components | (Wt. % Active) | (Wt. % Active) | (Wt. % Active) | (Wt. % Active) |
| Tallow/Coco Soap (80/20) | 69.5 | 69.5 | 69.5 | 69.5 |
| Stearic/Coco Fatty Acids | 6.0 | 6.0 | 6.0 | 6.0 |
| (1:1 ratio) | | | | |
| Glycerin | 3.5 | 3.5 | 3.5 | 3.5 |
| Salt (NaCl) | 1.0 | 1.0 | 1.0 | 1.0 |
| ALPHA-STEP ® PC-48 | 5.0 | | | |
| ALPHA-STEP ® BSS-45 | | 5.0 | | |
| ALPHA-STEP ® PS-65 | | | 5.0 | |
| ALPHA-STEP ® PS-45 SF | | | | 5.0 |
| AMPHOSOL ® HCG | 5.0 | 5.0 | 5.0 | 5.0 |
| Water | 10.0 | 10.0 | 10.0 | 10.0 |

TABLE 9

| | Formulation 23 | Formulation 24 | Formulation 25 | Formulation 26 |
|---|---|---|---|---|
| Components | (Wt. % Active) | (Wt. % Active) | (Wt. % Active) | (Wt. % Active) |
| Tallow/Coco Soap (80/20) | 74.5 | 77.5 | 78.5 | 74.5 |
| Stearic/Coco Fatty Acids | 6.0 | 6.0 | 6.0 | 6.0 |
| (1:1 ratio) | | | | |
| Glycerin | 3.5 | 3.5 | 3.5 | 3.5 |
| Salt (NaCl) | 1.0 | 1.0 | 1.0 | 1.0 |
| ALPHA-STEP ® BSS-45 | 2.5 | 1.0 | 0.5 | 5.0 |
| AMPHOSOL ® HCG | 2.5 | 1.0 | 0.5 | |
| Water | 10.0 | 10.0 | 10.0 | 10.0 |

TABLE 10

| | Formulation 27 | Formulation 28 | Formulation 29 | Formulation 30 |
|---|---|---|---|---|
| Components | (Wt. % Active) | (Wt. % Active) | (Wt. % Active) | (Wt. % Active) |
| Tallow/Coco Soap (80/20) | 69.5 | 69.5 | 69.5 | 69.5 |
| Stearic/Coco Fatty Acids | 6.0 | 6.0 | 6.0 | 6.0 |
| (1:1 ratio) | | | | |
| Glycerin | 3.5 | 3.5 | 3.5 | 3.5 |
| Salt (NaCl) | 1.0 | 1.0 | 1.0 | 1.0 |
| ALPHA-STEP ® BSS-45 | 5.0 | 5.0 | 5.0 | 5.0 |
| LATHANOL ® LAL | 5.0 | | | |
| Powder | | | | |
| Disodium Laureth | | 5.0 | | |
| Sulfosuccinate | | | | |
| NINOL ® COMF | | | 5.0 | |
| AMMONYX ® HCDO | | | | 5.0 |
| Water | 10.0 | 10.0 | 10.0 | 10.0 |

TABLE 11

| | Formulation 31 | Formulation 32 | Formulation 33 | Formulation 34 |
|---|---|---|---|---|
| Components | (Wt. % Active) | (Wt. % Active) | (Wt. % Active) | (Wt. % Active) |
| Tallow/Coco Soap (80/20) | 69.5 | 69.5 | 69.5 | 69.5 |
| Stearic/Coco Fatty Acids | 6.0 | 6.0 | 6.0 | 6.0 |
| (1:1 ratio) | | | | |
| Glycerin | 3.5 | 3.5 | 3.5 | 3.5 |
| Salt (NaCl) | 1.0 | 1.0 | 1.0 | 1.0 |
| ALPHA-STEP ® BSS-45 | 5.0 | 5.0 | 5.0 | 5.0 |
| BIO-TERGE ® AS-40 HA | 5.0 | | | |
| STEOL ® CS-370 | | 5.0 | | |
| STEPANOL ® P-30 | | | 5.0 | |
| STEPANOL ® MG | | | | 5.0 |
| Water | 10.0 | 10.0 | 10.0 | 10.0 |

TABLE 12

| | Formulation 35 | Formulation 36 | Formulation 37 | Formulation 38 |
|---|---|---|---|---|
| Components | (Wt. % Active) | (Wt. % Active) | (Wt. % Active) | (Wt. % Active) |
| Tallow/Coco Soap (80/20) | 69.5 | 69.5 | 69.5 | 64.5 |
| Stearic/Coco Fatty Acids | 6.0 | 6.0 | 6.0 | 6.0 |
| (1:1 ratio) | | | | |
| Glycerin | 3.5 | 3.5 | 3.5 | 3.5 |
| Salt (NaCl) | 1.0 | 1.0 | 1.0 | 1.0 |
| ALPHA-STEP ® BSS-45 | 5.0 | 5.0 | 5.0 | 7.5 |
| AMPHOSOL ® HCG | | | | 7.5 |
| NINOL ® C-5 | 5.0 | | | |
| AMPHOSOL ® 160 C | | 5.0 | | |
| MAPROSYL ® 30 | | | 5.0 | |
| Water | 10.0 | 10.0 | 10.0 | 10.0 |

TABLE 13

| | Formulation 39 | Formulation 40 | Formulation 41 | Formulation 42 |
|---|---|---|---|---|
| Components | (Wt. % Active) | (Wt. % Active) | (Wt. % Active) | (Wt. % Active) |
| Tallow/Coco Soap (80/20) | 59.5 | 49.5 | 61.5 | 17.0 |
| Stearic/Coco Fatty Acids | 6.0 | 6.0 | 6.0 | 25.0 |
| (1:1 ratio) | | | | |
| Glycerin | 3.5 | 3.5 | 3.5 | |
| Salt (NaCl) | 1.0 | 1.0 | 1.0 | |
| ALPHA-STEP ® PC-48 | | | 4.0 | 4.0 |
| ALPHA-STEP ® BSS-45 | 10.0 | 15.0 | | |
| ALPHA-STEP ® PS-45 SF | | | 14.0 | 45.0 |
| AMPHOSOL ® HCG | 10.0 | 15.0 | | 4.0 |
| Water | 10.0 | 10.0 | 10.0 | 5.0 |

TABLE 14

| | Formulation 43 | Formulation 44 | Formulation 45 | Formulation 46 |
|---|---|---|---|---|
| Components | (Wt. % Active) | (Wt. % Active) | (Wt. % Active) | (Wt. % Active) |
| Tallow/Coco Soap (80/20) | 64.5 | 77.5 | 74.5 | 69.5 |
| Stearic/Coco Fatty Acids | 6.0 | 6.0 | 6.0 | 6.0 |
| (1:1 ratio) | | | | |
| Glycerin | 3.5 | 3.5 | 3.5 | 3.5 |
| Salt (NaCl) | 1.0 | 1.0 | 1.0 | 1.0 |
| ALPHA-STEP ® BSS-45 | 5.0 | 1.0 | 2.5 | 5.0 |
| AMPHOSOL ® HCG | 5.0 | | | |
| LATHANOL ® LAL | 5.0 | | | |
| Powder | | | | |
| Disodium Laureth | | 1.0 | 2.5 | 5.0 |
| Sulfosuccinate | | | | |
| Water | 10.0 | 10.0 | 10.0 | 10.0 |

TABLE 15

| | Formulation 47 | Formulation 48 | Formulation 49 | Formulation 50 |
|---|---|---|---|---|
| Components | (Wt. % Active) | (Wt. % Active) | (Wt. % Active) | (Wt. % Active) |
| Tallow/Coco Soap (80/20) | 64.5 | 59.5 | 49.5 | 69.5 |
| Stearic/Coco Fatty Acids | 6.0 | 6.0 | 6.0 | 6.0 |
| (1:1 ratio) | | | | |
| Glycerin | 3.5 | 3.5 | 3.5 | 3.5 |
| Salt (NaCl) | 1.0 | 1.0 | 1.0 | 1.0 |
| ALPHA-STEP ® BSS-45 | 7.5 | 10.0 | 15.0 | 1.0 |
| AMPHOSOL ® HCG | | | | 9.0 |
| Disodium Laureth | 7.5 | 10.0 | 15.0 | |
| Sulfosuccinate | | | | |
| Water | 10.0 | 10.0 | 10.0 | 10.0 |

TABLE 16

| | Formulation 51 | Formulation 52 | Formulation 53 | Formulation 54 |
|---|---|---|---|---|
| Components | (Wt. % Active) | (Wt. % Active) | (Wt. % Active) | (Wt. % Active) |
| Tallow/Coco Soap (80/20) | 69.5 | 69.5 | 69.5 | |
| Tallow/Coco Soap (60/40) | | | | 69.5 |
| Stearic/Coco Fatty Acids | 6.0 | 6.0 | 6.0 | 6.0 |
| (1:1 ratio) | | | | |
| Glycerin | 3.5 | 3.5 | 3.5 | 3.5 |
| Salt (NaCl) | 1.0 | 1.0 | 1.0 | 1.0 |
| ALPHA-STEP ® BSS-45 | 9.0 | 4.0 | 6.0 | 5.0 |
| AMPHOSOL ® HCG | 1.0 | 6.0 | 4.0 | 5.0 |
| Water | 10.0 | 10.0 | 10.0 | 10.0 |

TABLE 17

| | Formulation 55 | Formulation 56 | Formulation 57 | Formulation 58 |
|---|---|---|---|---|
| Components | (Wt. % Active) | (Wt. % Active) | (Wt. % Active) | (Wt. % Active) |
| Tallow/Coco Soap (80/20) | | 69.5 | 69.5 | 69.5 |
| Tallow/Coco Soap (85/15) | 69.5 | | | |
| Stearic/Coco Fatty Acids | 6.0 | 6.0 | 6.0 | 6.0 |
| (1:1 ratio) | | | | |
| Glycerin | 3.5 | 3.5 | 3.5 | 3.5 |
| Salt (NaCl) | 1.0 | 1.0 | 1.0 | 1.0 |
| ALPHA-STEP ® BSS-45 | 5.0 | | | 5.0 |
| ALPHA-STEP ® PS-65 | | | | |
| ALPHA-STEP ® PS-45 SF | | 5.0 | | |
| AMPHOSOL ® HCG | 5.0 | | | |
| ALPHA-STEP ® DS-85 | | 5.0 | | |
| ALPHA-STEP ® BSS-85 | | | 5.0 | |
| Alkylpolyglycoside | | | | 5.0 |
| Sodium Lauroyl Lactylate | | | 5.0 | |
| Water | 10.0 | 10.0 | 10.0 | 10.0 |

TABLE 18

| | Formulation | Formulation | Formulation |
|---|---|---|---|
| | 59 | 60 | 61 |
| | (Wt. % | (Wt. % | (Wt. % |
| Components | Active) | Active) | Active) |
| Tallow/Coco Soap (80/20) | 69.5 | 69.5 | 70.81⁵ |
| Stearic/Coco Fatty Acids | 6.0 | 6.0 | 4 |
| (1:1 ratio) | | | |
| Glycerin | 3.5 | 3.5 | 3.5 |
| Salt (NaCl) | 1.0 | 1.0 | 1.0 |
| ALPHA-STEP ® BSS-45 | 5.0 | 5.0 | 6.7 |
| Alkylpolyglycoside | | 5.0 | |
| Sodium Lauroyl Lactylate | 5.0 | | |
| Sodium Lauryl Sulfate | | | 3.3 |
| Inactives | | | 1.69 |
| Water | 10.0 | 10.0 | 9 |

Note
⁵Sodium Tallow/Coco Soap (80/20)

### Example 9

### Manufacturing Procedure

The formulations disclosed in Tables 1-18 may be prepared according to the following procedure. Below is the manufacturing procedure for a single exemplary formulation:

Crutching Step. About 127.3 parts of a mix containing: 31.67% water, 46.7% 85/15 tallow/coconut (T/CN) soap, 0.43% sodium chloride, 2.75% glycerin, 4.69% coconut free fatty acid (CNFA), 9.46% of sodium coconut alpha sulfo Methyl ester 1:1 Mono/di ratio paste, and 3.93% of NINOL® CMP or LMP are added to a crutcher in the indicated order. Mix the product at about 85 ° C. to about 90° C.

Vacuum Drying Step. The crutcher mix is then vacuum dried at approximately 50 mm Hg absolute pressure to reduce the moisture content of the mix to about 10% and to plod this soap into noodles.

Amalgamating Step. The soap noodles are weighed and placed in a batch amalgamator. To about 97.0 parts noodles in the amalgamator are added: 0.50 part TiO₂, 2.0 parts perfume, 0.1% BHT, 0.1% Citric Acid, 0.15 part colorant solution, and 0.15 part of a solution which contains ca. 40% EDTA. The combined ingredients are mixed thoroughly.

Milling Step. Three-roll soap mills are set up with all rolls at about 85° F. to about 105° F. (about 29° C. to about 41° C.). The mixture from the amalgamator is passed through the mills several times to obtain a homogeneous mix. This is an intimate mixing step.

Plodding and Stamping Steps. A conventional plodder is set up with the barrel temperature at about 35° C. and the nose temperature at about 42° C. The plodder used is a dual stage twin screw plodder that allows for a vacuum of about 40 to about 65 mm Hg between the two stages. The soap log extruded from the plodder is typically round, and is cut into individual plugs. These plugs are then stamped on a conventional soap stamping apparatus to yield the finished toilet soap bar.

It has been discovered that the soap bars made from the above compositions possess surprising performance and processing advantages. These advantages are demonstrated below by the marring data, phase behavior and rheology/microstructure profile.

### Example 10

### Soap Bar Marring

Marring is the damage incurred by impact to a soap bar during handling and shipping. It is a well-known characteristic by which consumers rate a bar. Bar soap manufacturers prefer a soap formulation with low mar characteristics to reduce consumer rejection should the bars incur any damage or rough handling during shipping. The bars of the present technology show little damage when dropped compared to conventional soap bars. As an illustration of this, soap bars prepared according to the present technology are subjected to a test that quantitatively compares different bars by their marring characteristics.

Each sample is weighed and then dropped from a specific height to mar the bars. It was determined that exactly 7 feet would provide an extreme enough impact to clearly determine the marring characteristics of the bars. The bars would be dropped in a way that the small end of the bar would strike the ground to provide the most visible damage possible (striking perpendicular to the extrusion of the bars). The bars are then analyzed for their level of damage in the form of a dry-impact bar cracking scale. Using this scale the mar value of the bar is determined through ranking of the visible damage to the bar.

TABLE 19
The Dry-Impact Cracking Scale

| Mar Value | Visible characteristics |
|---|---|
| 0 | No cracks or chips, a smooth dent |
| 1 | Very fine spider cracks |
| 2 | Hair-line fracturing |
| 3 | Visible deep cracks with potential for chipping |
| 4 | Slight chipping along edge of damage |
| 5 | Noticeable chips from around area of impact |
| 6 | Obvious deforming/shattering of bar, large |
| | chunks broken off of bar |

The bar mar test method was analyzed for reproducibility. Samples are tested in triplicate to ensure reproducibility and determine the standard deviation. The average standard deviation of the mar values for the samples is 0.39, showing a high reproducible rate within a range of 1 on the dry-impact cracking scale.

The test method is used to determine the marring characteristics of several trial bars made according to the presently described technology, and several conventional commercial bars. Each bar is dropped from a height of 7 feet and the damage measured to calculate the total marring value of each sample.

The results summarized in Table 20 indicate that the trial bars according to the present technology show a marring value of zero, which is lower than any of the commercial conventional bars evaluated in the test. It is apparent that the present compositions provides a bar with lower mar than the conventional plain soap or combination bars.

TABLE 20
Marring Test Results

| Sample | Mar Mean Value |
|---|---|
| First Commercial US Combination | 4.66 |
| Bar | |
| Second Commercial US Combination | 3.33 |
| Bar | |
| Commercial Mexican Combination | 1.66 |
| Bar | |
| Formulation 3 (Table 2) | 0.33 |
| Formulation 5 (Table 3) | 0.0 |
| Formulation 6 (Table 3) | 0.0 |

### Example 11

### Viscosity & Rheology

It has also been surprisingly found that the presently disclosed soap bar compositions containing alpha sulfonated alkyl ester, sulfonated fatty acid, or mixtures thereof, in addition to polyhydric alcohol and electrolyte, are easier to process than conventional soap compositions. For example, soap bar compositions of the present technology are readily pumpable using standard soap bar production equipment, as compared to compositions prepared in the absence of alpha sulfonted alkyl ester, sulfonated fatty acid, or mixtures thereof, polyhydric alcohol and electrolyte.

While not being bound by any particular theory, it is believed that enhanced processability of the presently disclosed soap bar compositions is in part due to their rheology and viscosity characteristics; specifically, initial soap slurry compositions according to the present technology generally exhibit lower viscocity at lower temperature. Furthermore, formulations according to the present technology generally exhibit constant viscocity more quickly in shear tests. Table 21 illustrates the lowered viscocity of certain exemplary formulations of the present technology, compared to control samples without sulfonated fatty acid (SFA) and/or sulfonated alkyl ester (SME), or without polyhydric alcohol. Viscosity was measured in a continuous ramp test at constant shear rate of 2 1/s and at 70° C. with an AR-2000 rheometer from TA Instruments of New Castle, Del. A 4 cm plate-plate geometry was used for these tests. After shearing for 100 seconds and 300 seconds, the viscosity was recorded. Table 10 shows the viscosity results.

TABLE 21
Viscosities of SME Soap Slurries
from Constant Flow Measurement

| | Viscosity at 100 Sec | Viscosity at 300 Sec |
|---|---|---|
| Sample | (Pa · S) | (Pa · S) |
| Control Formulation A | 9.3 | 5.7 |
| Control Formulation B | 5.1 | 4.3 |
| Formulation 15 | 2.1 | 2.1 |
| Formulation 16 | 3.1 | 3.1 |
| Control Formulation C | 15.2 | 14.2 |
| Formulation 17 | 5.6 | 5.1 |
| Formulation 18 | 5.7 | 5.4 |
| Formulation 61 | 9.8 | 7.8 |

It is believed, while not being limited to any one theory, that lower viscocity is at least in part attributable to a lower phase transition temperature of the present compositions from an undesirable hexagonal microstructure to a desirable lamellar microstructure. It is believed that compositions exhibiting a lamellar microstructure generally have a lower shear viscocity than compositions with a hexagonal microstructure. Table 22 illustrates the phase morphology of several embodiments of the present technology, compared to control samples without alpha sulfonated alkyl ester and/or sulfonated fatty acid (SME/SFA), or polyhydric alcohol. Tested embodiments of the presently disclosed technology exhibited a primarily lamellar microstructure at approximately 70° C., compared to control formulations without SME/SFA or polyhydric alcohol, which exhibited a primarily hexagonal microstructure at about 70° C. Hexagonal microstructures have high viscosity and yield stress, and are known to be more difficult to process. The control formulations exhibited phase transition temperatures between about 75° C. to about 90° C., while the formulations according to the present technology exhibited phase transition temperatures between about 57° C. to about 65° C. These tests also indicate a synergistic relationship in compositions utilizing or containing both SME/SFA and polyhydric alcohol—namely, compositions containing both SME/SFA and polyhydric alcohol exhibit more desirable viscosity and microstructure than compositions containing only one.

TABLE 22
Microstructure of SME Soap Slurries

| | | | Phase Transition |
|---|---|---|---|
| | | | Temperature |
| | | | (Approximate) (hexagonal |
| | Phase at 70° C. | Texture at 70° C. | change to lamellar) |
| Control Formulation A | Hexagonal | Hexagonal Gel | 80° C. |
| (Without SME/SFA) | | | |
| Control Formulation B | Hexagonal | Hexagonal Gel | 90° C. |
| (Without Glycerin) | | | |
| Control Formulation C | Hexagonal | Hexagonal and | 75° C. |
| (Without SME/SFA) | | Mosaic | |
| Formulation 15 | Lamellar | Maltese crosses | 60° C. |
| | | and oily streak | |
| Formulation 16 | Lamellar/ | Maltese crosses | 60° C. |
| | isotropic | | |
| Formulation 17 | Lamellar | Maltese crosses | 57° C. |
| Formulation 18 | Lamellar | Maltese crosses | 62° C. |
| Formulation 61 | Lamellar | Maltese crosses | 65° C. |

It is also believed that the improved rheological and microstructural properties of the present compositions also may result in improved physical characteristics of a finished soap bar. For example, in a lamellar structure, water binds with the polar groups of surfactants and form in a sheet type highly ordered structured water phase. The water is distributed more evenly and is available uniformly as its structure recovery under shear is fast. This results into much better drying properties of lamellar soap melt. Due to uniform moisture distribution in the soap melt/slurry, there will be very few dry and moist spots in extruded bars. During storage or use these bars, they may not lose or absorb different amount of water causing the bar to develop cracks at the point of moisture gradient difference. Thus, the bar produced from a lamellar soap melt/slurry will have much more uniform evaporation of water over time and would display characteristics of much better elasticity.

Without being bound by any particular theory, it is believed that the preferred compositions can evenly distribute the bound water, making such water not easily available for evaporation under storage temperatures. As a result, very little crystallinity occurs in the finished bar, making it less susceptible to marring. This is another positive and desirable attribute of SME soap bar technology.

The invention and the manner and process of making and using it, are now described in such full, clear, concise and exact terms as to enable any person skilled in the art to which it pertains, to make and use the same. It is to be understood that the foregoing describes some embodiments of the invention and that modifications may be made therein without departing from the spirit or scope of the invention as set forth in the claims.

1-3. (canceled)

4. The composition of claim 21, wherein the soap mixture comprises between about 60% to about 95% by weight of the soap of tallow soap and between about 5% to about 40% by weight of the soap of coconut soap.

5. The composition of claim 21, comprising an alpha sulfonated alkyl ester having the formula: 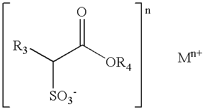
wherein R₃ is a C₆-C₂₂ hydrocarbyl group, an alkyl group, or a combination thereof, R₄ is a straight or branched chain C₁-C₆ hydrocarbyl group, an alkyl group, or combinations thereof, n is 1 or 2 and M is hydrogen, sodium, potassium, calcium, magnesium, ammonium, monoethanolammonium, diethanolammonium, triethanolammonium, a derivative thereof, or a mixture thereof.

6. The composition of claim 21, comprising a sulfonated fatty acid having the formula: 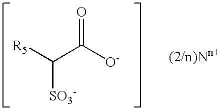
wherein, R₅ is a C₆-C₂₂ hydrocarbyl group, an alkyl group, or a combination thereof, n is 1 or 2 and N is hydrogen, sodium, potassium, calcium, magnesium, ammonium, monoethanolammonium, diethanolammonium, triethanolammonium, a derivative thereof, or a mixture thereof.

7. The composition of claim 21, further comprising up to about 10% of an alkanolamide.

8-16. (canceled)

17. The composition of claim 21, comprising between about 3% to about 16% by weight water.

8. The composition of claim 21, wherein the composition has a phase transition temperature from hexagonal to lamellar of less than about 65° C. in slurry.

19-20. (canceled)

21. A soap bar composition comprising:
(a) from about 40% to about 94% by weight of a mixture of a tallow soap and a coconut soap;
(b) from about 1% to about 15% by weight of a coconut, stearic fatty acid;
(c) a surfactant mixture of:
(i) from about 2% to about 8% by weight of a mixture of alpha sulfonated alkyl ester and sulfonated fatty acid in a ratio of about 10:1 to about 1:10 of alpha sulfonated alkyl ester to sulfonated fatty acid; and
(ii) from about 1% to about 10% by weight of a salt of alkyl sulfate;
(d) between about 0.5% to about 2% by weight of sodium chloride;
(e) between about 0.5% to about 6% by weight glycerin; and
(f) between about 3% to about 22% by weight water; and
wherein a substantial portion of the soap bar composition exhibits a lamellar microstructure at about 70° C. in slurry.

22. The soap bar composition of claim 21, wherein the surfactant mixture comprises about 10% by weight of the soap bar composition.

23. The soap bar composition of claim 22, wherein the salt of alkyl sulfate is sodium lauryl sulfate.

24. The soap bar composition of claim 23, wherein the mixture of alpha sulfonated alkyl ester and sulfonated fatty acid comprises about 6.7% by weight of the soap bar composition and the sodium lauryl sulfate comprises about 3.3% by weight of the soap bar composition.
